(19) United States
(12) Patent Application Publication
(10) Pub. No.: US 2008/0119542 A1
(43) Pub. Date: May 22, 2008

(54) METHODS OF USING ARTEMISININ-LIKE COMPOUNDS TO PREVENT OR DELAY THE APPEARANCE OF CANCER

(75) Inventors: Henry C. Lai, Seattle, WA (US); Narendra P. Singh, Lynnwood, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: UNIVERSITY OF WASHINGTON, Seattle, WA (US)

(21) Appl. No.: 11/948,560

(22) Filed: Nov. 30, 2007

Related U.S. Application Data
(63) Continuation of application No. 10/457,079, filed on Jun. 6, 2003, now abandoned.
(60) Provisional application No. 60/386,928, filed on Jun. 6, 2002.

(51) Int. Cl. A61K 31/357; A61P 35/00

(52) U.S. Cl. 514/450

(57) ABSTRACT

The invention provides methods for preventing or delaying the development of cancer by administering free radical-generating agents to a subject. Representative free radical-generating agents include endoperoxide compounds, such as endoperoxides bearing sesquiterpene compounds such as artemisinin and its analogs, arteflene and its analogs, 1,2,4-trioxanes and 1,2,4,5-tetraoxanes. Intracellular iron concentrations may be enhanced by the administration of iron salts or complexes.

### CROSS-REFERENCE TO RELATED APPLICATION

This application is a continuation of U.S. application Ser. No. 10/457,079, filed Jun. 6, 2003, which claims the benefit of U.S. Provisional Application No. 60/386,928, filed Jun. 6, 2002.

### FIELD OF THE INVENTION

The present invention relates to methods for preventing or delaying the appearance of cancer by administering a free radical-generating agent to a subject.

### BACKGROUND OF THE INVENTION

Artemisinin is a sesquiterpene lactone isolated from the plant Artemisia annua L., extracts of which has been used to treat malaria for at least 1600 years. The artemisinin molecule contains an endoperoxide bridge that reacts with an iron atom to form free radicals, causing cell death. The anti-malarial action of artemisinin is due to its reaction with intra-parasitic heme to generate free radicals, causing cell death. Cancer cells have a significantly higher influx of iron than normal cells. Accordingly, it has been shown that artemisinin and artemisinin analogs are cytotoxic against established tumors and tumor cell lines (see, e.g., Woerdenbag et al. (1993) J. Nat. Prod. 56(6):849-56; Lai & Singh (1995) Cancer Lett. 91:41-6; Efferth et al. (2001) Int. J. Oncol. 18:767-73; Li et al. (2001) Bioorg. Med. Chem. Lett. 11:5-8; Singh & Lai (2001) Life Sci. 70:49-56; Efferth et al. (2002) Biochem. Pharmacol. 64:617-23; Efferth et al. (2002) Blood Cells, Molecules & Diseases 28(2):160-8; Sadava et al. (2002) Cancer Lett. 179:151-6).

Many analogs of artemisinin and other compounds containing an endoperoxide bridge that are biologically active have been described (see, e.g., U.S. Pat. No. 5,180,840; U.S. Pat. No. 5,216,175; U.S. Pat. No. 5,225,427; Cumming et al. (1998) J. Med. Chem. 41(6):952-64; Posner et al. (1999) J. Med. Chem. 42:300-4; Li et al. (2001) Bioorg. Med. Chem. Lett. 11:5-8; Wu et al. (2001) Eur. J. Med. Chem. 36:469-79; Posner et al. (2003) J. Med Chem 46:1060-5). Analogs of artemisinin that have been used in the treatment of malaria include dihydroartemisinin, artemether, artesunate, arteether, propylcarbonate dihydroartemisinin and artelinic acid.

Artemisinin is a relatively safe drug with few and minor side effects even at high doses. Oral doses of 70 mg/kg/day for 6 days has been used in humans for malaria treatment. No apparent adverse side effects were observed after treatment of a cancer patient with artesunate (oral dose of 50 mg per day; intramuscular dose of 60 mg/day, over a period of 9 months) (Singh & Verma (2002) Arch. Oncol. 10(4):279-80). Artemisinin and artemisinin analogs have also been used in the treatment of skin conditions such as psoriasis, blistering skin diseases, viral warts, mulluscum contagiosum, and hemorrhoids (see, e.g., U.S. Pat. No. 4,978,676; U.S. Pat. No. 5,219,880). Artemisinin and artemisinin analogs have also been used for malaria prophylaxis.

Cancer generally develops from the predisposition of a single cell to proliferate in an uncontrolled manner. There is a need in the art for methods for inhibiting cells predisposed to develop into cancer, thereby preventing or delaying the onset of cancer. The present invention addresses this need.

### SUMMARY OF THE INVENTION

The present invention provides methods for preventing or delaying the appearance of cancer. The methods comprise administering to a subject an amount of a free radical-generating agent that is effective to prevent or delay the appearance of cancer. In some embodiments, the free radical-generating agent is a compound containing an endoperoxide bridge. Typically, the endoperoxide compound is selected from the group consisting of sesquiterpene lactones and alcohols, carbonates, esters, ethers, and sulfonates thereof, arteflene, 1,2,4-trioxanes, and 1,2,4,5-tetraoxanes. The endoperoxide compound may be a compound of the formula:

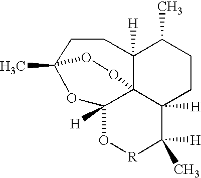

wherein R is

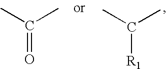

where R₁ is hydrogen, hydroxyl, alkyl, or has the formula:

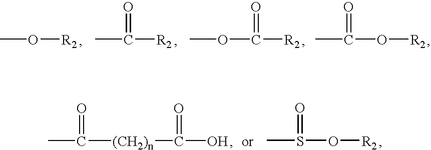

wherein R₂ is alkyl or aryl and n is 1 to 6, or a pharmaceutically acceptable salt thereof. Exemplary endoperoxide compounds useful in the practice of the invention include, but are not limited to, artemisinin, dihydroartemisinin, artemether, arteether, artesunate, artelinic acid, and dihydroartemisinin propyl carbonate.

Free radical-generating agents also include compounds that do not contain an endoperoxide bridge but that can react with iron to form free radicals, for example carbon-based free radicals. The source of free radical-generating agents may be natural (e.g., isolated from plants), synthetic, or semi-synthetic. For example, the free radical-generating agents may be produced by expressing the enzymes for the relevant synthetic pathways in a microbial host (see, e.g., Martin et al. (2003) Nature Biotechnol., published online: 1 Jun. 2003, doi: 10.1038/nbt833).

In some embodiments, the methods further comprise administering an effective amount of an intracellular iron-enhancing agent to the subject. Exemplary intracellular iron-enhancing compounds are iron salts and complexes, including ferrocholinate, ferroglycine sulfate, dextran iron complex, peptonized iron, iron sorbitex, ferric oxide, saccharated iron, holoferritins, and holotransferrins.

The methods of the invention are applicable to any mammalian subject, such as a human subject. The methods of the invention provide a significant delay in the appearance of cancer in subjects. Thus, in some embodiments, subjects exposed to a carcinogen that were treated with a free radical-generating agent according to the methods of the invention remained cancer-free for twice as long as control animals that were not treated with a free radical-generating agent.

Moreover, the methods of the invention provide a reduced likelihood of the appearance of cancer. Thus, in some embodiments, none of the subjects exposed to a carcinogen that were treated with a free radical-generating agent according to the methods of the invention developed cancer, compared to 43% of control animals that were not treated with a free radical-generating agent.

In a further aspect, the invention provides kits that include a free radical-generating agent and instructions for using the free radical-generating agent for delaying or preventing the appearance of cancer in a subject. In some embodiments, the kits of the invention may further include iron-enhancing agents.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a graphical representation of the percentage of animals that developed tumors after 1-38 weeks from exposure to a carcinogen with no treatment (shown as “♦”) or after weekly oral administration of artemisinin (shown as “▪”), as described in EXAMPLE 1.

FIG. 2 shows a graphical representation of the percentage of animals that developed tumors after 1-10 weeks from exposure to a carcinogen with no treatment (shown as “♦”) or after treatment with artemisinin mixed in food (shown as “▪”), as described in EXAMPLE 2.

### DETAILED DESCRIPTION OF THE PREFERRED EMBODIMENT

In one aspect the present invention provides methods for preventing or delaying the appearance of cancer, comprising administering to a subject an amount of a free radical-generating agent that is effective to prevent or delay the appearance of cancer. The methods of the invention are applicable to any mammalian subject, such as a human subject or an animal subject. As used herein, the term “preventing or delaying the appearance of cancer” refers to the prevention of the appearance of cancer and/or the delay in the appearance of cancer that would occur in the absence of administration of a free radical-generating agent according to the invention. The term “appearance of cancer” refers to the time point at which a cancer is clinically manifest or detectable by any method suitable for the detection and diagnosis of cancer. Thus, the appearance of cancer may be postponed for a definite period of time or indefinitely. The prevention or delay in the appearance of cancer may occur, for example, by killing of pre-cancerous cells or cells genetically or environmentally inclined to abnormal or uncontrolled cellular proliferation, by preventing the division of these cells, or by preventing the abnormal or uncontrolled division of these cells. By preventing or delaying the appearance of cancer, the free radical-generating agents may prolong the period before the appearance of a cancer and/or reduce the likelihood of the appearance of cancer.

The term “free radical-generating agent” refers to any agent that can react with iron or iron-containing compounds to generate free radicals, such as carbon-based free radicals. In some embodiments, the free radical-generating agent is an endoperoxide compound. The term “endoperoxide compound” refers to a compound that possesses an endoperoxide bridge that reacts in the presence of iron and iron-containing compounds to form free radicals. Endoperoxide compounds may also form free radicals in the presence of copper and manganese Representative endoperoxide compounds are set forth herein, although it will be apparent that other endoperoxide compounds will be useful for this purpose.

Exemplary endoperoxide compounds useful in the practice of the invention are artemisinin and its analogs and other compounds containing an endoperoxide bridge that can react with iron and iron-containing compounds to form free radicals. Accordingly, suitable endoperoxide compounds include sesquiterpene lactones and alcohols, carbonates, esters, ethers, and sulfonates thereof, arteflene, 1,2,4-trioxanes, and 1,2,4,5-tetraoxanes. Suitable endoperoxide bearing sesquiterpene compounds of the present invention comprise compounds of the formula:

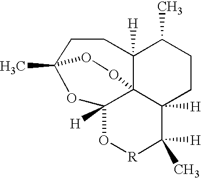

wherein R is

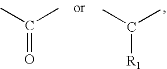

where R₁ is hydrogen, hydroxyl, alkyl, or has the formula:

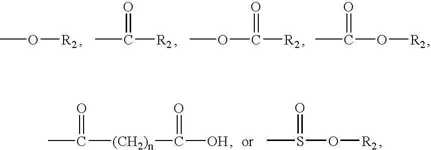

where R₂ is alkyl or aryl and n is 1 to 6, and the pharmaceutically acceptable salts thereof. As used herein, the term “alkyl” means lower alkyl having from 1 to 6 carbon atoms, preferably 1 to 4 carbon atoms. Alkyl groups of the invention may be straight-chain or branched-chain groups. The term “aryl” refers to monocyclic and polycyclic aromatic groups containing from 4 to 14 backbone carbon or heteroatoms, and includes both carbocyclic aryl groups and heterocyclic aryl groups. Carbocyclic aryl groups are aryl groups in which all ring atoms are carbon. Heterocyclic aryl groups have from 1 to 4 heteroatoms as ring atoms, with the remainder of the ring atoms being carbon. Representative aryl groups include, for example, phenyl and benzyl. Pharmaceutically acceptable salts include the alkali or alkaline earth metal salts, preferably sodium or potassium.

For example, endoperoxide compounds of the invention include artemisinin, where R is

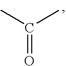

dihydroartemisinin (R₁=—OH), artesunic acid (R₁═—OCO(CH₂)₂CO₂H), and artesunate, artemether (R₁═—OCH₃), and arteether (R₁═—OC₂H₅). Other representative endoperoxide compounds of the invention include artelinic acid, dihydroartemisinin propyl carbonate, arteflene (Ro. 42-1611) and its analogs (Biirgen et al. (1994) Sixth Int. Cong. Infect. Dis. Abst. 427, p. 152, Prague), 1,2,4-trioxanes (Peters et al. (1993) Ann. Trop. Med. Parasit. 87(1):9-16) and 1,2,4,5-tetraoxanes (Vennerstrom et al. (1992) J. Med. Chem. 35(16):3023-3027). Other suitable structural analogs of artemisinin are described in, for example, U.S. Pat. Nos. 5,216,175 and 5,180,840; Cumming et al. (1998) J. Med. Chem. 41(6):952-64; and PCT patent applications WO 97/01548, WO 99/33461, and WO 00/42046.

Amounts of the free radical-generating agents that are effective to inhibit cells predisposed to develop into cancer range up to the maximally tolerated dosage, but the concentrations are not critical and may vary widely. The precise amounts employed will vary, of course, depending on the compound, route of administration, physical condition of the patient and other factors. The daily dosage may be administered as a single dosage or may be divided into multiple doses for administration.

The amount of the free radical-generating agent actually administered will be a prophylactic effective amount, which term is used herein to denote the amount needed to produce a substantial preventative or delaying effect on the appearance of cancer. Effective doses may be extrapolated from dose-response curves derived from in vitro or animal model test systems. The animal model is also typically used to determine a desirable dosage range and route of administration. Such information is used to determine useful doses and routes for administration in humans or other mammals. The determination of an effective dose is well within the capability of those skilled in the art. Thus, the amount actually administered will be dependent upon the individual subject, and will preferably be an optimized amount such that the desired effect is achieved without significant side-effects.

Prophylactic efficacy and possible toxicity of the free radical-generating agents, such as endoperoxide compounds, can be determined by standard pharmaceutical procedures, in cell cultures or experimental animals (e.g., ED₅₀, the dose therapeutically effective in 50% of the population; and LD₅₀, the dose lethal to 50% of the population). The dose ratio between therapeutic and toxic effects is the therapeutic index, and it can be expressed as the ratio LD₅₀ to ED₅₀. Free-radical generating agents that exhibit large therapeutic indices, for example a therapeutic index of at least about 60, are particularly suitable in the practice of the methods of the invention. The data obtained from cell culture assays and animal studies may be used in formulating a range of dosage for use in humans or other mammals. The dosage of free radical-generating agents lies preferably within a range of circulating concentrations that include the ED₅₀ with little or no toxicity. The dosage typically varies within this range depending upon the dosage form employed, sensitivity of the subject, and the route of administration. Thus, optimal amounts will vary with the method of administration, and will generally be in accordance with the amounts of conventional medicaments administered in the same or a similar form. Topical or oral administration, for instance, may typically be done from once to three times a day.

For endoperoxide compounds such as artemisinin and its analogs, good results may be obtained using formulations comprising the compounds at levels of from about 0.1 to about 20 mg per kilogram of body weight per day, such as from about 0.5 to about 15 mg per kilogram of body weight per day, such as from about 1 to about 10 mg per kilogram of body weight per day. An exemplary daily oral dosage of endoperoxides such as artemisinin, artemether, or arteether for an adult human subject is from about 20 mg to about 200 mg, such as from about 40 mg to about 180 mg, or from about 80 mg to about 160 mg. Similar dosages may be used for other free radical-generating agents.

Administration of the free radical-generating agents of the invention is accomplished by any effective route, for example, orally, as described in EXAMPLES 1 and 2. The free radical-generating agents may also be administered parenterally. Methods of administration include topical, inhalational, buccal, intraarterial, subcutaneous, intramedullary, intravenous, intranasal, intrarectal, intraocular administration, and other conventional means. The free radical-generating agents may be formulated into a composition that additionally contains suitable pharmaceutically acceptable carriers, including excipients and other compounds that facilitate administration of the free radical-generating agent to a mammalian subject. Further details on techniques for formulation and administration may be found in the latest edition of “Remington's Pharmaceutical Sciences” (Maack Publishing Co, Easton Pa.).

Free radical-generating agents for oral administration may be formulated using pharmaceutically acceptable carriers well known in the art, in dosages suitable for oral administration, as described in EXAMPLES 1 and 2. Such carriers enable the free radical-generating agents to be formulated as tablets, pills, dragees, capsules, liquids, gels, syrups, slurries, suspensions, etc., suitable for ingestion by a subject. Free radical-generating agents for oral use may be formulated, for example, in combination with a solid excipient, optionally grinding the resulting mixture, and processing the mixture of granules, after adding suitable additional compounds, if desired, to obtain tablets or dragee cores. Suitable excipients include carbohydrate or protein fillers. These include, but are not limited to, sugars, including lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins, such as gelatin and collagen. If desired, disintegrating or solubilising agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage).

Free radical-generating agents for oral administration may be formulated, for example, as push-fit capsules made of gelatin, as well as soft, sealed capsules made of gelatin and a coating such as glycerol or sorbitol. Push-fit capsules may contain endoperoxide compounds mixed with filler or binders such as lactose or starches, lubricants such as talc or magnesium stearate, and, optionally, stabilizers. In soft capsules, the free radical-generating agents may be dissolved or suspended in suitable liquids, such as fatty oils, liquid paraffin, or liquid polyethylene glycol with or without stabilizers. For example, the free radical-generating agents, such as endoperoxide compounds like artemether, arteether, artemisinin, or other artemisinin analogs, may be dissolved in an oil such as soybean oil, olive oil, or peanut oil. Suitable soybean oils include, but are not limited to, soybean oil from the soybean Glycine Soya Bentham (e.g., Shanghai Number 2 Oil Factory, cat. no. 91102).

Compositions for parenteral administration include aqueous solutions of one or more free radical-generating agents. For injection, the endoperoxide compounds of the invention may be formulated in aqueous solutions, such as in physiologically compatible buffers such as Hank's solution, Ringer's solution, or physiologically buffered saline. Aqueous injection suspensions may contain substances, which increase the viscosity of the suspension, such as sodium carboxymethyl cellulose, sorbitol, or dextran. Additionally, suspensions of free radical-generating agents may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents, which increase the solubility of the free radical-generating agents to allow for the preparation of highly concentrated solutions.

For topical or nasal administration, penetrants appropriate to the particular barrier to be permeated are typically used in the formulation. Examples of these are 2-pyrrolidone, N-methyl-2-pyrrolidone, dimethylacetamide, dimethyl-formamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur. For topical administration, the composition may be in the form of a transdermal ointment or patch for systemic delivery of the compound and may be prepared in a conventional manner (see, e.g., Barry, Dermatological Formulations (Drugs and the Pharmaceutical Sciences—Dekker); Harrys Cosmeticology (Leonard Hill Books).

For rectal administration, the compositions may be administered in the form of suppositories or retention enemas. Such compositions may be prepared by mixing the free radical-generating agent with a suitable non-irritating excipient that is solid at ordinary temperatures but liquid at the rectal temperature and will therefore melt in the rectum to release the drug. Suitable excipients include, but are not limited to, cocoa butter and polyethylene glycols.

The amounts of each of these various types of additives will be readily apparent to those skilled in the art, optimal amounts being the same as in other, known formulations designed for the same type of administration. Stratum corneum penetration enhancers, for example, will typically be included at levels within the range of about 0.1% to about 15%.

Compositions containing the free radical-generating agents of the present invention may be manufactured in a manner similar to that known in the art (e.g., by means of conventional mixing, dissolving, granulating, dragee-making, levigating, emulsifying, encapsulating, entrapping or lyophilizing processes). The compositions may also be modified to provide appropriate release characteristics, e.g., sustained release or targeted release, by conventional means (e.g., coating).

Compositions containing free radical-generating agents may be provided as a salt and can be formed with many acids, including but not limited to hydrochloric, sulfuric, acetic, lactic, tartaric, malic, succinic, etc. Salts tend to be more soluble in aqueous or other protonic solvents than are the corresponding free base forms.

After such compositions formulated to contain free radical-generating agents and an acceptable carrier have been prepared, they can be placed in an appropriate container and labeled for use.

The free radical-generating agents of the invention may be administered alone, or in combination with one or more additional therapeutically active agents. For example, higher efficacy of endoperoxide compounds may be achieved, for example, by increasing oxygen tension, decreasing intake of antioxidants, and blockade of peroxidase and catalase by drugs such as miconazole. The effectiveness of free radical-generating agents may also be enhanced by administering an intra-cellular iron-enhancing compound. Accordingly, some embodiments of the invention additionally comprise administering an intra-cellular iron-enhancing agent. The free radical-generating agents, such as endoperoxide compounds, react with iron to form free radicals. It has been shown that administration of iron salts or the iron-carrying protein holotransferrin increases the susceptibility of cancer cells to artemisinin or dihydroartemisinin (Lai & Singh (1995) Cancer Lett. 91:41-46; Moore et al. (1995) Cancer Lett. 98:83-7; Singh & Lai (2001) Life Sci. 70:49-56; Sadava et al. (2002) Cancer Lett. 1179:151-6). According to the methods of some embodiments of the invention, the administration of an intracellular iron-enhancing agent increases the effectiveness of the free radical-generating agents for delaying or preventing the appearance of cancer.

The term “intracellular iron-enhancing agent” refers to any agent that is effective to increase the intracellular concentration of ferrous iron and include pharmaceutically acceptable iron salts and iron complexes. Iron salts useful in the practice of the invention include ferrous fumarate, ferrous sulfate, ferrous carbonate, ferrous citrate, ferrous gluconate and ferrous lactate. Iron complexes useful in the practice of the invention generally include pharmaceutically acceptable complexes comprising iron, such as, for example, ferrocholinate, ferroglycine sulfate, dextran iron complex, peptonized iron, iron sorbitex, ferric oxide and saccharated iron, as well as iron complexed with iron binding proteins and glycoproteins, such as the holoferritins and holotransferrins.

The concentrations of intracellular iron-enhancing agents used in the present invention will generally range up to the maximally tolerated dose for a particular subject and agent, which will vary depending on the agent, subject, disease condition and other factors, as described above. Dosages ranging from about 1 to about 20 mg of iron per kilogram of subject body weight per day will generally be useful for this purpose. In one embodiment, the intracellular iron-enhancing agents may be administered before administration of the free radical-generating agent.

The methods of the invention provide a significant delay in the appearance of cancer in a subject. Thus, in some embodiments, subjects exposed to a carcinogen that were treated with a free radical-generating agent according to the methods of the invention remained cancer-free at least for about twice as long as control animals that were not treated with a free radical-generating agent, as described in EXAMPLE 1.

Moreover, the methods of the invention provide a reduced likelihood in the appearance of cancer. Thus, fewer subjects exposed to a carcinogen that were treated with a free radical-generating agent developed cancer than control animals that were not treated with an endoperoxide compound, as described in EXAMPLES 1 and 2. In some embodiments, none of the subjects exposed to a carcinogen that were treated with a free radical-generating agent according to the methods of the invention developed cancer, compared to 43% of control animals that were not treated with a free radical-generating agent, as described in EXAMPLE 2.

In a further aspect, the invention provides kits that include one or more free radical-generating agents and instructions for using the free radical-generating agents for delaying or preventing the appearance of cancer. The instructions may be conditions under which administration of the free radical-generating agents are likely to be especially beneficial, for example, the presence of a familial history of cancer, previous incidences of cancer, or exposure to a carcinogen. The instructions may also provide recommendations for administration, such as dosages. In some embodiments, the kits of the invention may further include iron-enhancing agents and instructions for their use in combination with the endoperoxide compounds.

The following examples merely illustrate the best mode now contemplated for practicing the invention, but should not be construed to limit the invention.

### Example 1

This Example describes a representative method for preventing or slowing the development of cancer in rats exposed to a carcinogen by administering artemisinin by weekly oral intubations.

Materials and Methods: Female Sprague-Dawley rats were administered a single intragastric dose of the carcinogen 7,12-dimethylbenz[a]anthracene (DMBA; Sigma Chemicals, St Louis, Mo.; 50 mg/kg body weight) suspended in olive oil (Sigma Chemicals, St Louis, Mo.; 25 mg/ml). Animals were randomly assigned to two treatment groups to receive either weekly oral intubations of artemisinin (Holley Pharmaceuticals, Fullerton, Calif.; 10 mg/kg at 10 mg/ml of olive oil) or olive oil (control). Artemisinin treatment began a week after DMBA administration. Animals were palpated for the presence of mammary tumors once weekly. The time and site of detection and size were recorded for each tumor in an animal.

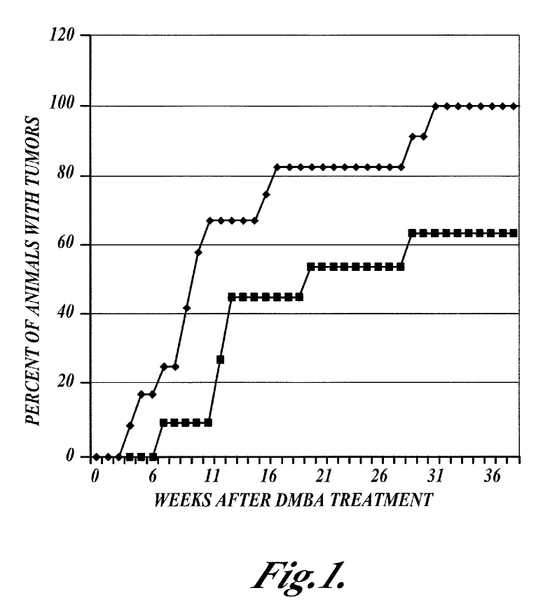

Results: 6 weeks after the carcinogen treatment, none of the artemisinin-treated animals had developed a tumor, whereas 17% of the control animals had tumors. 10 weeks after the carcinogen treatment, 9% of the artemisinin-treated animals had developed tumors, compared to 58% of control animals. 20 weeks after the carcinogen treatment, 54% of the artemisinin-treated animals had developed tumors, compared to 83% of control animals. 38 weeks after the carcinogen treatment, 64% of the artemisinin-treated animals had developed tumors, compared to 100% of control animals. These results are shown in Table 1 and FIG. 1.

TABLE 1
Tumor Development in Animals Treated Weekly with Artemisinin

Percentage of Animals with Tumors

| Week | Artemisinin-Treated | Control |
|---|---|---|
| 6 | 0 | 17 |
| 7 | 9 | 25 |
| 9 | 9 | 42 |
| 10 | 9 | 58 |
| 11 | 9 | 67 |
| 12 | 27 | 67 |
| 13 | 45 | 67 |
| 16 | 45 | 75 |
| 17 | 45 | 83 |
| 20 | 54 | 83 |
| 29 | 64 | 92 |
| 31 | 64 | 100 |
| 38 | 64 | 100 |

Thus, the weekly artemisinin-treatment delayed the appearance of tumors in carcinogen-treated animals. Moreover, fewer animals developed tumors compared to the controls.

### Example 2

This Example describes a representative method for preventing or slowing the development of cancer in rats exposed to a carcinogen by providing artemisinin mixed in food.

Materials and Methods: Female rats were similarly treated with the carcinogen DMBA, as described in EXAMPLE 1. The rats were randomly assigned to two treatment groups. Artemisinin (0.05%) was mixed in the food of one group of animals starting the day immediately after DMBA treatment. Daily intake of artemisinin, based on the amount of food eaten each day, was estimated to be approximately 10 mg/kg/day. The other group of animals (controls) was given regular rat food.

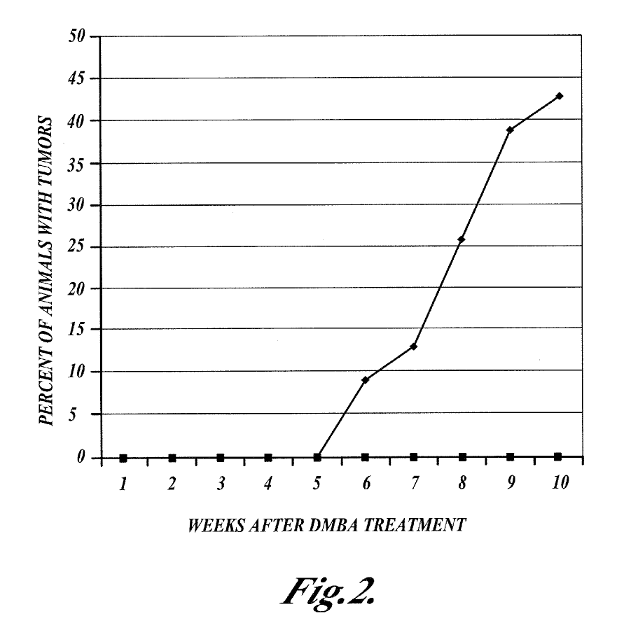

Results: Ten weeks after the carcinogen treatment, none of the artemisinin-fed animals had developed a tumor, whereas 43% of the control animals had developed tumors, as shown in Table 2 and FIG. 2.

TABLE 2
Tumor Development in Animals Fed Artemisinin

Percentage of Animals with Tumors

| Week | Artemisinin-Fed | Control |
|---|---|---|
| 6 | 0 | 9 |
| 7 | 0 | 13 |
| 8 | 0 | 26 |
| 9 | 0 | 39 |
| 10 | 0 | 43 |

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for preventing or delaying the appearance of cancer, comprising administering to a subject an amount of a free radical-generating agent that is effective to prevent or delay the appearance of cancer.

2. The method of claim 1, wherein the free radical-generating agent is an endoperoxide compound selected from the group consisting of sesquiterpene lactones and alcohols, carbonates, esters, ethers, and sulfonates thereof, arteflene, 1,2,4-trioxanes, and 1,2,4,5-tetraoxanes.

3. The method of claim 2, wherein the endoperoxide compound is a compound of the formula: 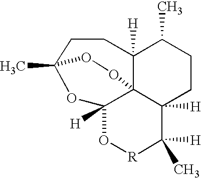
wherein R is 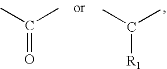
where R₁ is hydrogen, hydroxyl, alkyl, or has the formula: 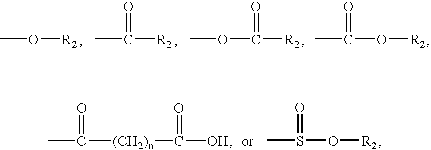
wherein R₂ is alkyl or aryl and n is 1 to 6, and the pharmaceutically acceptable salts thereof.

4. The method of claim 2, wherein the endoperoxide compound is a sesquiterpene compound selected from the group consisting of artemisinin, dihydroartemisinin, artemether, arteether, artesunate, artelinic acid, and dihydroartemisinin propyl carbonate.

5. The method of claim 4, wherein the endoperoxide compound is artemisinin.

6. The method of claim 4, wherein the endoperoxide compound is artemether.

7. The method of claim 2, wherein from about 0.1 to about 20 mg/kg of the endoperoxide compound is administered to the subject per day.

8. The method of claim 2, wherein the endoperoxide compound is administered orally or topically.

9. The method of claim 1, wherein the free radical-generating agent is artemisinin, the subject is a human subject, and the artemisinin is orally administered in the form of a powder, a tablet, or a capsule at dosage of between about 0.1 to about 20 mg/kg per day.

10. The method of claim 1, wherein administering the free radical-generating agent to the subject provides a delay in the appearance of cancer.

11. The method of claim 1, wherein administering the free radical-generating agent to the subject provides a reduced likelihood of the appearance of cancer.

12. The method of claim 1 further comprising administering an effective amount of an intracellular iron-enhancing agent to the subject.

13. The method of claim 12, wherein the intracellular iron-enhancing agent is selected from the group consisting of ferrocholinate, ferroglycine sulfate, dextran iron complex, peptonized iron, iron sorbitex, ferric oxide, saccharated iron, holoferritins, and holotransferrins.

14. The method of claim 12, wherein the intracellular iron-enhancing agent is administered to the subject prior to administering the free radical-generating agent.

15. A kit, comprising a free radical-generating agent and instructions for using the free radical-generating agent for preventing or delaying the appearance of cancer.

16. The kit of claim 15, wherein the free radical-generating agent is an endoperoxide compound selected from the group consisting of sesquiterpene lactones and alcohols, carbonates, esters, ethers, and sulfonates thereof, arteflene, 1,2,4-trioxanes, and 1,2,4,5-tetraoxanes.

17. The kit of claim 16, wherein the endoperoxide compound is artemisinin.

18. The kit of claim 16, wherein the endoperoxide compound is artemether.

19. The kit of claim 15 further comprising an iron-enhancing agent and instructions for using the iron-enhancing agent.
